SEQUENCE LISTING (14 sequences)

SEQ ID NO: 1 - protein, 19 aa, Asterina pectinifera
EKYCDDDFHMAVFRTCAVS

SEQ ID NO: 2 - protein, 24 aa, Asterina pectinifera
SEYSGIASYCCLHGCTPSELSVVC

SEQ ID NO: 3 - DNA, 351 bp, Asterina pectinifera
atgacaagcaacaaccgccatctcttccaggcaacttgcctagttctcctccttctacacgctgccttccacggtggagccctcggtgagaagtactgcgatgacgatttccacatggcggtgttccggacgtgcgcggtcagcaagcggagccagccggggatgagccttagcgacgtgttgaccatgaaccgctttcgaggtcacaacattaaacgaagcatcgacagcacacttgaagacaacgcctttttcatgagcggtttggagaagagatctgaatacagcggcatcgcctcgtactgttgccttcacggatgcacgcccagtgaattgtccgtcgtctgctaa

SEQ ID NO: 4 - protein, 116 aa, Asterina pectinifera
MTSNNRHLFQATCLVLLLLHAAFHGGALGEKYCDDDFHMAVFRTCAVSKRSQPGMSLSDVLTMNRFRGHNIKRSIDSTLEDNAFFMSGLEKRSEYSGIASYCCLHGCTPSELSVVC

SEQ ID NO: 5 - DNA, 23 bp, Artificial sequence
  primer
  misc_feature (1)..(23): Y means T or C.
tgygaygaygayttycayatggc

SEQ ID NO: 6 - DNA, 23 bp, Artificial sequence
  primer
  misc_feature (1)..(23): Y means T or C, and N means A, C, G or T.
atggcngtnttycgnacntgcgc

SEQ ID NO: 7 - DNA, 23 bp, Artificial sequence
  primer
  misc_feature (1)..(23): N means A, C, G or T, and R means G or A.
ggngtrcanccrtgnagrcaaca

SEQ ID NO: 8 - DNA, 20 bp, Artificial sequence
  primer
  misc_feature (1)..(20): N means A, C, G or T, and R means G or A
tcrctnggngtrcanccrtg

SEQ ID NO: 9 - DNA, 32 bp, Artificial sequence
  primer
cagcacctattacacacctcgaaagcggttca

SEQ ID NO: 10 - DNA, 31 bp, Artificial sequence
  primer
cgaagcatcgacagcacacttgaagacaacg

SEQ ID NO: 11 - DNA, 21 bp, Artificial sequence
  primer
ttccggacgtgcgcggtcagc

SEQ ID NO: 12 - DNA, 21 bp, Artificial sequence
  primer
cggacgtgcgcggtcagcaag

SEQ ID NO: 13 - DNA, 30 bp, Artificial sequence
  primer
gcaacagtacgaggcgatgccgctgtattc

SEQ ID NO: 14 - DNA, 31 bp, Artificial sequence
  primer
cagtacgaggcgatgccgctgtattcagatc

(19) United States
(12) Patent Application Publication
(10) Pub. No.: US 2008/0096251 A1
(43) Pub. Date: Apr. 24, 2008

(54) Invertebrate-Derived Gonadotropic Hormone and its Synthesis

(76) Inventors: Yoshitaka Nagahama, Aichi (JP); Michiyasu Yoshikuni, Aichi (JP); Masatoshi Mita, Tokyo (JP); Minoru Isobe, Aichi (JP)

Correspondence Address: DRINKER BIDDLE & REATH (DC), 1500 K STREET, N.W., SUITE 1100, WASHINGTON, DC, 20005-1209, US

(21) Appl. No.: 11/661,140

(22) PCT Filed: Aug. 25, 2005
(86) PCT No.: PCT/JP05/15458

§ 371 Date: Feb. 26, 2007

(30) Foreign Application Priority Data
Aug. 27, 2004 (JP) 2004-247779

(51) Int. Cl. C07K 14/575; C07K 19/00; C12P 21/02

(52) U.S. Cl. 435/69.4; 530/350; 536/23.1

(57) ABSTRACT

The structure of gonadotropic hormone of an invertebrate (Asterina pectinifera) was elucidated. The gonadotropic hormone is a peptide with molecular weight 4500 to 4900 composed of two subunits, wherein the structure of the protein has the structure bound by SS bridges formed between SH residues of cysteines contained in the subunits. Mixing and oxidizing these two subunits after their synthesis enables to give a peptide (gonadotropic hormone) with gonad-stimulating activity.

### FIELD OF THE INVENTION

The present invention relates to a gonadotropic hormone derived from invertebrates such as Asterina pectinifera and the like, and its synthesis.

### BACKGROUND OF THE INVENTION

There are two kinds of vertebrate-derived gonadotropic hormones, which control development of genital grand, and development, maturation and release of eggs and sperms. There has been considerable progress in the elucidation on gonadotropic hormones derived from vertebrates such as fishes and the like. The structural similarity of these hormones is well conserved from fishes to humans and these hormones are protein hormones with heterodimeric structure comprising an α subunit and a β subunit.

Vertebrate-derived gonadotropic hormones are confirmed to have cross-species effect of gonadotropic stimulation (non-patent reference 1, patent reference 1 and 2) for vertebrates but not for invertebrates.

Invertebrate-derived gonadotropic hormones have not been so much elucidated as vertebrate-derived ones. A gonadotropic hormone (GSS) was extracted from a radial nerve of Asterina pectinifera, i.e. an invertebrate, and was confirmed to induce spawning from an ovarian piece (non-patent reference 2 and 3). It is reported that the gonadotropic hormone causes ovarian follicular cells surrounding eggs in an ovary to synthesize and to secrete oocyte maturation-inducing hormone (1-methyl adenine) directly affecting to eggs (non-patent reference 4).

Patent reference 1: Japanese Patent No. 2967945.

Patent reference 2: Japanese Patent Application Public Disclosure No. H06-107689

Non-patent reference 1: Ed., The Japanese society for comparative endocrinology, “Biological Science of Hormone 5, Hormone and Reproduction (II)”, Japan Scientific Societies Press, pp. 41-47, 1979.

Non-patent reference 2: Ed., The Japanese society for zoology, “Problems in Modern Zoology 4, Oocytes and Sperms”, Tokyo University Press, pp. 21-37, 1975.

Non-patent reference 3: Shirai H., Gonad-Stimulating and Maturation-Inducing Substance, “Method in Cell Biology” Academic Press, vol. 27, pp. 73-88, 1986.

Non-patent reference 4: Mita M. & Nagahama Y., Involvement of G-proteins and adenylate cyclase in the action of gonad-stimulating substance on starfish ovarian follicle cells, Developmental Biology, 1991, 144, 262-8.

### Problems to be Solved by the Invention

Since gonadotropic hormones have maturation-stimulating effects, they are importantly applied to cultivation of the source living organism. The present invention is based on the groundbreaking elucidation of the structure of an invertebrate-derived gonadotropic hormone and is prospectively applied to cultivation of invertebrates such as shrimps, crabs, seashells and the like.

### Means to Solve the Problems

Present inventors successfully analyzed the structure of a gonad-stimulating hormone secreted from nerve cells of Asterina pectinifera. The inventors elucidated for the first time that the gonadotropic hormone is a peptide with molecular weight of 4500 to 4900 comprising subunits of molecular weight 2000 to 2400 and 2400 to 2600 and that the structure is composed by SS bridges constructed between SH residues of cysteines contained in the two subunits, and confirmed that the peptide obtained by mixing and oxidation of the synthesized two subunits has the gonad-stimulating activity. The result analyzed by the present inventors may open new possibilities of wide range of applications as to enable mass production of the hormone and others.

Namely, the present invention is an invertebrate-derived gonadotropic hormone comprising the following two peptides, wherein SS bridges are formed between 6 cysteines of the two peptides:

(a) A peptide having an amino acid sequence of SEQ ID NO: 1, or an amino acid sequence comprising a deletion, substitution or addition of one or several amino acids (i.e. 2 to 3) in said amino acid sequence except amino acids 4 and 16 (Cys) of said amino acid sequence, and having a gonad-stimulating activity,

(b) A peptide having an amino acid sequence of SEQ ID NO: 2, or an amino acid sequence comprising a deletion, substitution or addition of one or several (i.e. 2 to 3) amino acids in said amino acid sequence except amino acids 10, 11, 15 and 24 (Cys) of said amino acid sequence, and having a gonad-stimulating activity.

Also, the present invention is an invertebrate-derived gonadotropic hormone prepared by mixing and oxidizing the following two peptides:

(a) A peptide having an amino acid sequence of SEQ ID NO: 1, or an amino acid sequence comprising a deletion, substitution or addition of one or several (i.e. 2 to 3) amino acids in said amino acid sequence (preferably amino acids except Cys) and having a gonad-stimulating activity,

(b) A peptide having an amino acid sequence of SEQ ID NO: 2, or an amino acid sequence comprising a deletion, substitution or addition of one or several (i.e. 2 to 3) amino acids in said amino acid sequence (preferably amino acids except Cys) and having a gonad-stimulating activity.

These two peptides could be obtained by purifying (a) a nerve organ removed from an invertebrate or (b) a certain organ reported to have gonad-stimulating activity, based on invertebrate gonad-stimulating activity as an indicator.

Furthermore, the present invention is a method for preparing peptides with gonad-stimulating activity comprising mixing and oxidizing the above two peptides. These two peptides could be obtained by any method, i.e. they may be chemically synthesized or may be obtained by the use of genetic engineering.

The present invention is DNA coding any one of above two peptides ((c) and (d)). Peptides are extracted from hosts or culture medium, wherein the hosts are transformed by a vector containing the above two DNA, cultured and grew in a culture medium. A peptide with gonad-stimulating activity could be prepared by mixing and oxidizing the peptides.

Furthermore, the present invention is DNA, which contains DNA coding the above two peptides ((c) and (d)), with more than 70% homologous to the nucleotide sequence of SEQ ID NO: 3. Peptides with gonad-stimulating activity could be prepared by culturing and growing host transformed by a vector containing the DNA. Moreover, peptides could be extracted from host or its culture medium of the host, wherein the peptides could be mixed and oxidized.

### BRIEF DESCRIPTION OF THE DRAWINGS

FIG. 1 is a photograph showing the criteria of gonad-stimulating activity. The right side shows that eggs are spawned and gonad-stimulating activity is present.

FIG. 2 shows the profile of high performance liquid chromatography for purifying gonadotropic hormone.

FIG. 3 shows the profile of high performance liquid chromatography for purifying gonadotropic hormone.

FIG. 4 shows the profile of microdose high performance liquid chromatography for purifying gonadotropic hormone.

FIG. 5 shows the profile of microdose high performance liquid chromatography for purifying gonadotropic hormone.

FIG. 6 shows the result of analysis of purified gonadotropic hormone (heterodimer) by a mass spectrometer.

FIG. 7 shows the result of analysis of subunits of gonadotropic hormone by a mass spectrometer

FIG. 8 shows the profile of high performance liquid chromatography for purifying synthesized gonadotropic hormone.

### DETAILED DESCRIPTION OF THE INVENTION

The DNA sequence of invertebrate-derived gonadotropic hormone gene of the present invention comprises nucleotide sequence (351 bases) of SEQ ID NO: 3, DNA coding above two peptides ((c) and (d)), or a nucleotides sequence which is more than 70% homologous to nucleotide sequence of SEQ ID NO: 3.

The report of human genome analysis project by human genome consortium (Science 2001, 291 (5507), 1304-1351) proposes on the criterion of automatic gene annotation in genome sequence by a computer program (Otto system), wherein the criterion to specify a gene region of a genome based on the homology to the polynucleotide sequence of a known genetic information should be more than 92% identity. The same report proposes the homology criterion of polynucleotide on 70% for searching genomes increased by gene duplication (later evolution could introduce gene mutation and could accompany partial changes of nucleotide sequence). Therefore, a nucleotide sequence with more than 70% homologous, preferably more than 92%, to SEQ ID NO: 3 in the present invention may function as an invertebrate gonadotropic hormone gene, which is applied to closely related but different animal species.

The amino acid sequence of the invertebrate gonadotropic hormone of the present invention comprises 116 amino acids shown in SEQ ID NO: 4 or an amino acid sequence wherein one or several (e.g. 2˜3) amino acids (preferably amino acids except Cys) are deleted, substituted or added in the amino acid sequence. In the amino acid sequence (SEQ ID NO: 4), the sequence from amino acid 1, methionine (M), to amino acids 29, glycine (G), is a signal sequence, that from amino acids 30, glutamine (E), to amino acids 48, serine (S), is the subunit A (GSS-A), that from amino acids 49, lysine (K) to amino acids 92, arginine (R), is the subunit C (GSS-C) and that from amino acids 93, serine (s) to amino acids 116, cysteine (C), is the subunit B (GSS-B).

The invertebrate gonadotropic hormone of the present invention contains a peptide formed 1 to 1 binding of GSS-A and GSS-B, wherein the peptide comprises SS bridges oxidatively formed between cysteines (amino acids 4 and 16 of SEQ ID NO: 1 and amino acids 10, 11, 15 and 24 of SEQ ID NO: 2) contained in each peptide. The possibility of the binding of SS bridges between GSS-A and GSS-B is maximum 12 ways.

The heterodimeric structure with bridge structure between GSS-A and GSS-B peptides may be constructed as a gonadotropic hormone after removal of the signal sequence from the peptide chain synthesized based on the gene followed by final removal of GSS-C portion from the bridged structure. Still furthermore, the GSS-C portion may also have a physiological activity according to the recent research issue.

Since all the invertebrate gonadotropic hormones of the present invention are secreted from nerve tissues, the source of invertebrate gonadotropic hormones from other species than Asterina pectinifera was limited to nerve tissues of the source species. If other animal species are reported to have gonad-stimulating activity in other tissues and organs, the tissues and organs could be used as sources for extraction.

Invertebrates involve many useful marine species like coelenterata such as corals, echinodermata such as sea-urchins and sea-slugs, mollusc such as octopuses and calamary, and crustocea such as shrimps and crabs.

The peptides could be purified from above sources for extraction by the use of gonad-stimulating activity as a marker.

The purification could be used by any methods and liquid chromatography and aqueous two-phase partition method could be used. However, high performance liquid chromatography is preferably used for highly purification. Size exclusion chromatography, ion exchange chromatography or reverse phase chromatography could be used as columns. Thus separated fractions by the purification method are put under selection by gonad-stimulating activity as a marker.

Gonad-stimulating activity could be measured by the following methods:

Those individuals, whose eggs are highly sensitive to oocyte maturation-inducing hormone, are selected from matured invertebrate female individuals and are separated as test individuals.

Ovaries are excised from test individuals into seawater and are cut in small pieces. The assay plate for multiple test samples is filled with 200 μl seawater. Two μl of a test sample for gonad-stimulating activity is diluted to 200 folds by mixing with 398 μl seawater. The half portion (200 μl) is diluted to two fold by mixing with 200 μl seawater in the assay plate. The half portion (200 μl) is further diluted to two fold by mixing with 200 μl seawater in the assay plate. Repeating of the procedure results in preparation of two fold dilution series from 200-folds dilution to 102,400-folds dilution. A small piece of ovary is added to each dilution sample and is allowed to rest at 25° C. The gonad-stimulating activity is evaluated by spawning of matured eggs from a contracted small piece of ovary after 1 hr. The relative gonad-stimulating activity is assessed by the degree of dilution.

Gonadotropic hormone has multiple functions, i.e. long and short periods (long-term: development of gonad, short-term: induction of spawning). Since the assay depends on the short-term function, the test individuals should bear matured oocytes with capability of egg production. It is preferable to collect test individuals at their early spawning period and to keep alive in a laboratory aquarium. All individuals collected are not always so much matured as producing eggs. Furthermore, if individuals are ever capable of egg production, their degree of maturation is subtly different and usually the sensitivity against a hormone varies with the individual. Therefore, it is necessary for assaying gonad-stimulating activity to collect matured individuals (those bearing oocytes with capability of egg production) and, moreover, to prepare several individuals with similar sensitivity against a hormone.

Gonadotropic hormone stimulates ovarian follicle cells, wherein the follicle cells secrete another hormone “oocyte maturation-inducing hormone”, which causes oocytes to induce spawning. Since the oocyte maturation-inducing hormone directly affects to oocytes, one of the short term functions of gonadotropic hormone is to induce production of the oocyte maturation hormone. Commercially available oocyte maturation-inducing hormone could be used to assess the sensitivity.

The above purification could be repeated for several times. As the results, single peptide with molecular weight 4500˜4900 could be obtained.

As shown in the following Examples, the peptide comprises two subunits (GSS-A and GSS-B), wherein the molecular weight of the subunits are 2000˜2400 and 2400˜2600. These subunits could be obtained by reducing the above peptide with high molecular weight. Various reductants are used for the reductive reaction and such mild reductants as dithiothreitol, 2-mercupto ethanol, thioglycolic acid, benzenthiol, parathiocresol and others are preferably used.

In contrast, mixing and oxidation of these two peptides leads to production of a peptide (i.e. the gonadotropic hormone of the present invention) with higher molecular weight than above described peptides. Since each of the two subunits contains cysteines, oxidation generates SS bridges between SH residues of cysteines contained in the subunits to bind these two subunits.

The above oxidation could be performed by the following oxidants other than the method shown in Examples:
o-Iodobenzoic acid, iodine.
Manganese dioxide, potassium permanganate
Hydrogen peroxide
Free oxygen (this is a reagent used in the following Examples. Oxidation reaction by free oxygen could be accelerated in the presence of trace amounts of ion and copper ions).

Furthermore, it is possible to increase the yield of the generation of right SS bridge between peptides by the presence of the following reagents, which are used for refolding of SS bridges of protein at the time of oxidation reaction of synthetic peptides:
Thioredoxin (a protein used for redox interaction in living system)
Protein disulfide isomerase (protein disulfide exchange enzyme in living organisms)
BMC, (+)-trans-1,2-bis(2-mercaptoacetamido)cyclohexane
4-Mercaptobenzeneacetate

The peptide of the present invention could be prepared by gene recombination, too. For example, a vector incorporated DNA (i.e bases 88-144 and 277-348 of SEQ ID NO: 3) coding amino acid sequence of SEQ ID NOs: 1 and 2 or amino acid sequence, wherein one or several (i.e. 2 to 3) amino acids except Cys in the amino acid sequences are deleted, substituted or added or a vector incorporated DNA, wherein the nucleotide sequence is more than 70% homologous to the nucleotide sequence of SEQ ID NO: 3 are prepared, are transduced into hosts to be transformed. Then, the transformed hosts are cultured and grown. The aimed peptides are purified from the hosts or the culture medium of the hosts. Obtained peptides are sometimes peptides with linkage between the two subunits (GSS-A and GSS-B). However, oxidation of obtained peptides (two subunits) in the presence of the above oxidants leads to those peptides with gonad-stimulating activity.

The methods for inducing maturation promotion and ovulation by the use of the obtained peptides with gonad-stimulating activity involve injecting these peptides directly into coelom or ovary of invertebrates, mixing to seawater in aquarium or mixing into foods.

The following Examples are provided to illustrate the present invention, but are not intended to limit the scope thereof.

In the following Examples, gonad-stimulating activity are examined by the following way:

Two to three individuals bearing oocyte with high sensitivity of a gonadotropic hormone (1-methyladenine) are selected from several tens of female individuals of matured Asterina pectinifera (or Asterias amurensis) and are separated as test individuals.

The selection was performed in the following way:

The oocyte maturation-inducing hormone was diluted to 6 different concentrations, i.e. 10⁻⁶ M, 3×10⁻⁷ M, 10⁻⁷ M, 3×10⁻⁸ M, 10⁻⁸ M, and 3×10⁻⁹ M, in seawater, wherein small fragment of ovary was added to the different concentrations of maturation-inducing hormone and was allowed to stand for 60 min at room temperature. Maturation of oocyte was examined under microscope after 60 min to see whether the structure of nuclei in an oocyte was degraded to prepare for next fertilization. The concentration of the oocyte maturation-inducing hormone at maturation was checked and those individuals, which were matured at less than 10⁻⁷ M, were used as test individuals.

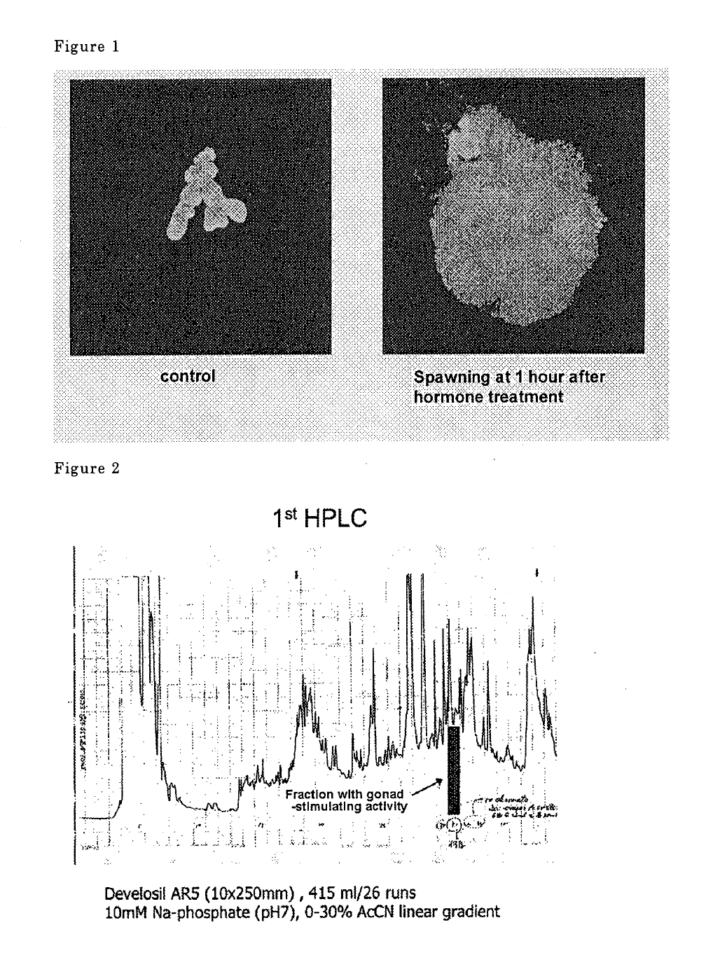

Ovaries (with tufted morphology) were removed from test individuals into seawater and were cut into about 5 mm fragments. The assay plate for multiple test samples is filled with 200 μl seawater. Two μl of a test sample for gonad-stimulating activity is diluted to 200 folds by mixing with 398 μl seawater. The half portion (200 μl) is diluted to two fold by mixing with 200 μl seawater in the assay plate. The half portion (200 μl) is further diluted to two fold by mixing with 200 μl seawater in the assay plate. Repeating of the procedure results in preparation of two fold dilution series from 200-folds dilution to 102,400-folds dilution. A small piece of ovary is added to each dilution sample and is allowed to rest at 25° C. The gonad-stimulating activity is evaluated by spawning of matured eggs from a contracted small piece of ovary after 1 hr (FIG. 1). The relative gonad-stimulating activity is assessed by the degree of dilution.

### EXAMPLE 1

Radial nerve tissues were removed from Asterina pectinifera by a forceps, were frozen on dry ice and were stored. 126.3 g wet weight nerve tissues were collected from 5550 starfishes.

Liquid nitrogen was filled in a mortar and radial nerve tissues stored in frozen state were ground into powder. The powdered nerve tissues were added with 600 mL of 10 mM ammonium acetate aqueous solution (containing proteinase inhibitors, i.e. 1 μM pepstatin, 0.5 mg/L leupeptin, 0.2 mM 4-(2-aminoethyl)benzenesulfonyl fluoride) in three separated times and were homogenized further to fine powders by a motor homogenizer (Physcotron). Extracts from the homogenized tissues was centrifuged at 22,500×g at 4° C. for 30 min and the supernatant was recovered. The pellet was homogenized with 200 mL of 10 mM ammonium acetate solution, was centrifuged at 22,500×g at 4° C. for 30 min. The supernatant was recovered and were mixed with the previous supernatant. The 22,500×g supernatant was ultracentrifuged at 100,000×g at 4° C. for 1 hr and the supernatant was recovered. The supernatant had gonad-stimulating activity.

After the obtained supernatant was frozen and dried, it was dissolved in 100 mL of 0.15 M ammonium carbonate. After undissolved residues were removed by centrifugation at 27,500×g at 4° C. for 30 min, the solution was applied to PD-10 desalting column (Amersham Biotech Co.) equilibrated with the ammonium carbonate solution and the high molecular weight effluent fraction (PD-10 fraction) with gonad-stimulating activity was recovered.

After the high molecular weight effluent fraction (PD-10 fraction) was frozen and dried, it was dissolved in 150 mL of 10 mM sodium phosphate (pH 7.0). 50 mL portions of the solution was applied to SephadexG-50 column (500 cm³) equilibrated with the sodium phosphate for total three times and the fraction with gonad-stimulating activity (the fraction except high molecular weight proteins) was recovered (G-50 fraction, the recovery was about 600 mL).

About 415 mL of G-50 fraction was applied to high performance liquid chromatography (Shimazu Corporation, Type LC-6AD) for 26 separated times. Then, the active fractions were eluted by linear concentration gradient from 10 mM sodium phosphate (pH 7.0) to 30% acetonitril/10 mM sodium phosphate by the use of Develosil RP-Aqueous AR5 column (10×250 mm, Nomura Science). The fractions with gonad-stimulating activity were eluted at around 18-19% of acetonitril concentrations (1^{st} HPLC fraction, FIG. 2).

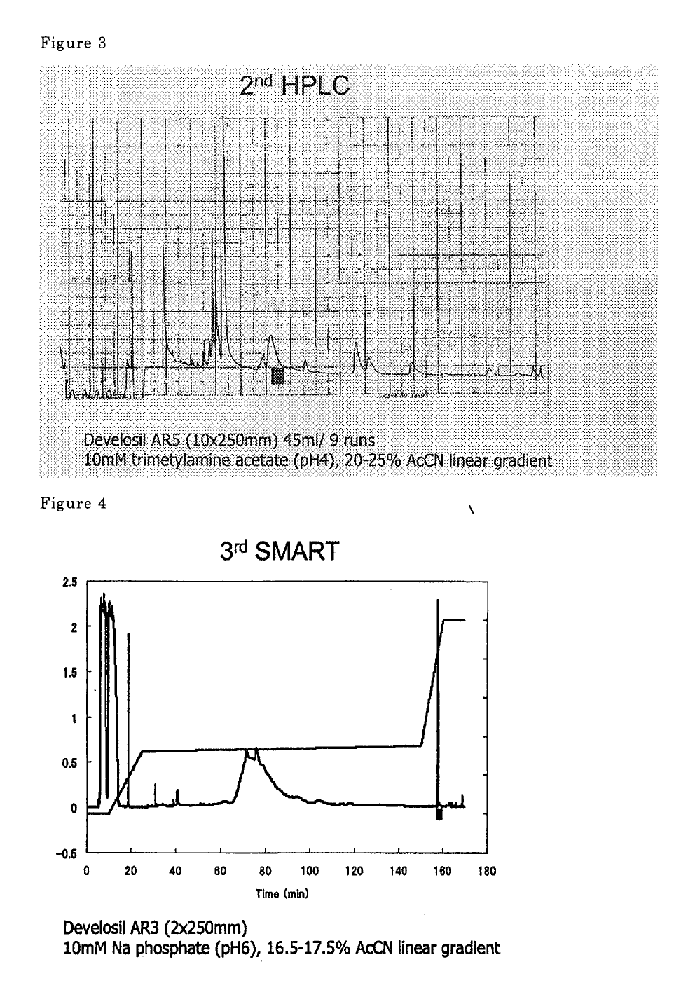

The 1^{st} HPLC fraction was concentrated to 45 mL under reduced pressure and the concentrate was applied to high performance liquid chromatography (Shimazu Corporation, Type LC-6AD) for 9 separated times. Then, the active fractions were eluted by linear concentration gradient from 20% acetonitril/10 mM trimethylamine acetate (pH 4.0) to 25% acetonitril/10 mM triethylamine acetate by the use of Develosil RP-Aqueous AR5 column (10×250 mm, Nomura Science). The fractions with gonad-stimulating activity were eluted at around 21-22% of acetonitril concentrations (2^{nd} HPLC fraction, FIG. 3).

The 2^{nd} HPLC fraction was concentrated under reduced pressure. The active fractions were eluted by linear concentration gradient from 16.5% acetonitril/10 mM sodium phosphate (pH 6.0) to 17.5% acetonitril/10 mM sodium phosphate (pH 6.0) by the use of Develosil RP-Aqueous AR3 column (2×250 mm, Nomura Science). The fractions with gonad-stimulating activity were eluted at around 17% of acetonitril concentrations (3^{rd} SMART fraction, FIG. 4).

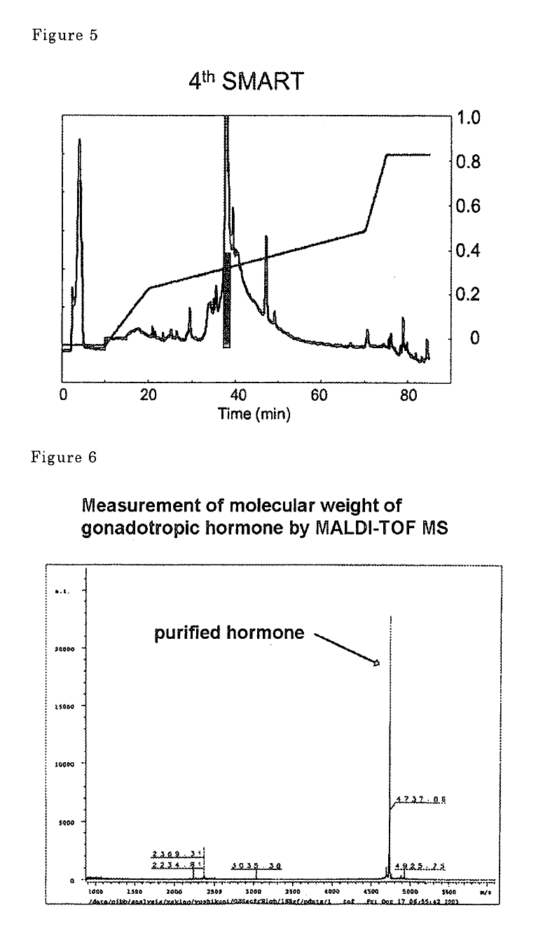

The active fractions were eluted by linear concentration gradient from 15% acetonitril/10 mM sodium phosphate (pH 6.0) to 30% acetonitril/10 mM sodium phosphate (pH 6.0) by the use of Develosil RP-Aqueous AR3 column (1.5×150 mm, Nomura Science). The fractions with gonad-stimulating activity were eluted at around 18% of acetonitril concentrations (4^{th} SMART fraction, FIG. 5).

A portion of the final purified fraction (4^{th} SMART fraction) was desalted by the use of Ziptip (Milipore Co.) and was analyzed by MALDI-TOF type mass spectrometer (Bruker Daltonics Co., ReflexIII type). The result showed a component with molecular weight of 4737. Furthermore, the final fraction was reduced with 50 mM dithiothreitol at room temperature for 1 hr and was analyzed again by the mass spectrometer. The result showed two signals with 2236 and 2507 instead of 4737. Moreover, the final fraction was treated with a reductant and was treated with an alkylating agent such as 0.2 M iodoacetamide at room temperature for 24 hrs. Since the molecular weights of peaks 2236 and 2507 were increased by 57 mass (corresponding to 1 SH residue) and 114 mass (corresponding to 2 SH residues), respectively, SH residues might be specifically alkylated.

Analysis of 4thSMART fraction by a protein sequencer (ABI, Procise Type 494HT) gave two signals, which indicate the mixture of two peptides. Since the above results indicate that the component with the gonad-stimulating activity contained in the final fraction was a polypeptide with molecular weight 4737 and the components split into two components by reduction, the active component has a heterodimeric structure comprising two subunit peptides with molecular weight 2236 and 2507, wherein the bridge of the dimeric structure is sensitive to a reductant and is shown to be SS bridge between cysteine residues.

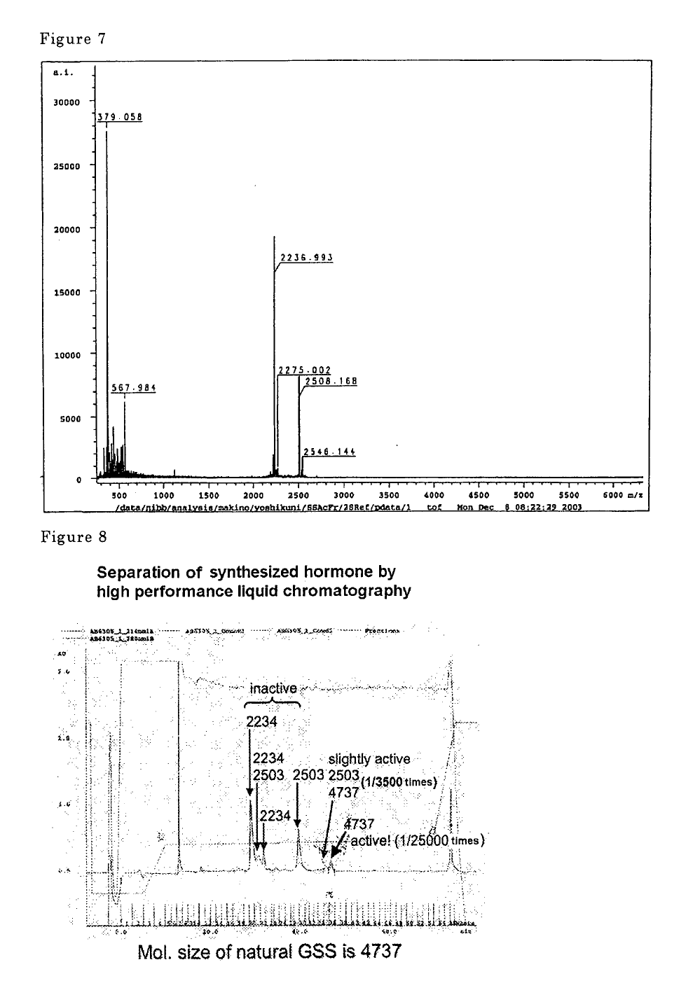

Amino acid analysis of the peptides mixture of molecular weight 2236 and 2507, obtained by reduction of the polypeptide with molecular weight 4737, was performed by mass spectrometry by the use of Q-TOF type mass spectrometer (Micromass Co.) (FIG. 6 and FIG. 7)

The amino acid sequences of the two peptides were:

GSS-A: EKYCDDDFHMAVFRTCAVS (SEQ ID NO: 1)(19 amino acid residues, Molecular weight 2236)

GSS-B: SEYSGIASYCCLHGCTPSELSVVC (SEQ ID NO: 2) (24 amino acid residues, Molecular weight 2507).

It was found from the results of mass spectrometer and protein sequencer analysis that these peptides are absent from any chemical modification (intra cellular post translational modification of protein and peptide) at the side chains and ends of their amino acids after biosynthesis in nerve cells of a starfish.

GSS gene was cloned on the base of amino acid sequences of GSS-A and GSS-B. Based on the amino acid sequences of GSS-A and GSS-B, 5′-primer DF1 (SEQ ID NO: 5) and 5′-primerDF2 (SEQ ID NO: 6), respectively; and 3′-primerDR1 (SEQ ID NO: 7) and 3′-primer DR2 (SEQ ID NO: 8), respectively, were synthesized.

Genome DNA was separated and purified from Asterina pectinifera testis by the use of QIAGEN® Genomic-tip.

A part of GSS gene sequence was amplified by nested PCR by the use of the genome sample as a template and by the use of synthesized degenerate primers and the nucleotide sequence was deciphered by a DNA sequencer. Base on the newly obtained sequence, 5′-primer GR (SEQ ID NO: 9) and 3′-primer GF (SEQ ID NO: 10) were synthesized. The 5′-upstream and 3′-downstream sequences of the DNA was deciphered by the use of the primers, the genome DNA and CLONTECH Genome Walker™ Kits. Based on the obtained sequences, 5′-primer MF1 (SEQ ID NO: 11) and 5′-primer MF2 (SEQ ID NO: 12); and 3′-primer MR1 (SEQ ID NO: 13) and 3′-primer MR2 (SEQ ID NO: 14) were synthesized.

Total RNA was separated and purified from nerves, ambulacral feet, hepatopancreas, testis and ovaries of Asterina pectinifera by the use of NIPPON GENE ISOGEN or QUIAGEN® QIAzol™. cDNA was synthesized from purified total RNA by the use of QIAGEN® Omniscript™ RT.

GSS cDNA sequence was amplified by the use of the cDNA as a template and synthesized the primers (5′-primer MF1, 5′-primer MF2,3′-primer MR1, 3′-primer MR2) by nested PCR and the GSS cDNA sequence was deciphered by the use of a DNA sequencer.

GSS gene shown as SEQ ID NO: 3 was thus obtained. The nucleotide sequence was translated to give amino acid sequence of SEQ ID NO: 4. The sequences of GSS-A and GSS-B analyzed by mass spectrometry and protein sequencer are located at amino acids 30-48 (GSS-A) and 93-116 (GSS-B) of the amino acid sequence of SEQ ID NO: 4.

Amino acids 1-29 of the amino acid sequence of SEQ ID NO: 4 are a signal sequence characteristic of secretory proteins and amino acids 49-92 of the amino acid sequence are an excised sequence (referred to as GSS-C) after biosynthesis of the amino acid sequence composed of 116 amino acids. KR sequence, which is cut specifically and enzymatically after biosynthesis, is located at both ends of GSS-C sequence.

### EXAMPLE 2

Based on the result of Example 1, two peptide chains (GSS-A and GSS-B) were synthesized (purity >99.5%).

Two synthesized peptides were dissolved in 20 mM Tris-buffer at concentration 0.4 mM or at 1 mM (each at equal number of moles), were reacted at room temperature for 3 days or for 20 days in the presence of oxidants with constant stirring. 99.999% oxygen gas or 0.1 M oxidized form of glutathion was used as an oxidant. After the reaction, the reactants were separated by a microdose high performance liquid chromatography and each peak fraction was assayed its gonad-stimulating activity.

Gonad-stimulating activity was detected in a relatively small peak fraction with peak area 4.2%. Furthermore, the molecular weight 4737 (GSS-A/B), which shows a complex structure and is the same to the molecular weight of natural hormone, was detected only in the peak fraction with gonad-stimulating activity according to the analysis of each peak by mass spectrometry. Other peaks without gonad-stimulating activity showed molecular weight 2236 or 2507 (FIG. 8).

The obtained peptides are assayed their gonad-stimulating activity. The results are shown in Table 1.

TABLE 1

EC50(nM)

| Peptide | Asterina pectinifera | Asterias amurensis |
|---|---|---|
| GSS-A | No activity | No activity |
| GSS-B | No activity | No activity |
| GSS-A/B | 0.6˜3.6 | 2˜10 |
| natural | 0.7˜4.0 | — |

Sole synthesized peptide (GSS-A or GSS-B) did not show gonad-stimulating activity and only the complex (GSS-A/B) with molecular weight 4737 synthesized by oxidation reaction showed the activity. The synthesized hormone (GSS-A/B) by oxidation reaction showed hormonal function not only in Asterina pectinifera but also in such related species as Asterias amurensis.

Present invention enables mass production of invertebrate gonad-stimulating hormone and made it possible to improve the amount of production of water invertebrates such as crabs, shrimps, sea urchins, sea cucumbers and shellfishes and to develop new valuable species.

Furthermore, artificial control of starfish numbers in the sea area of aquaculture by stocking with fertilized eggs of Asterina pectinifera and Asterias amurensis or their artificial proliferation may improve the quality of water by promoting biological decomposition of feedstuff accumulated in sea floor. Since these two starfish species are dominant species in the sea area of aquaculture and the habitat covers whole area from Hokkaido to Kyushu, considerable application could be expected.

1: An invertebrate-derived gonadotropic hormone comprising the following two peptides, wherein SS bridges are formed between 6 cysteines of the two peptides:
(a) A peptide having an amino acid sequence of SEQ ID NO: 1, or an amino acid sequence comprising a deletion, substitution or addition of one or several amino acids in said amino acid sequence except amino acids 4 and 16 of said amino acid sequence, and having a gonad-stimulating activity,
(b) A peptide having an amino acid sequence of SEQ ID NO: 2, or an amino acid sequence comprising a deletion, substitution or addition of one or several amino acids in said amino acid sequence except amino acids 10, 11, 15 and 24 of said amino acid sequence, and having a gonad-stimulating activity.

2: An invertebrate-derived gonadotropic hormone prepared by mixing and oxidizing the following two peptides:
(a) A peptide having an amino acid sequence of SEQ ID NO: 1, or an amino acid sequence comprising a deletion, substitution or addition of one or several amino acids in said amino acid sequence and having a gonad-stimulating activity,
(b) A peptide having an amino acid sequence of SEQ ID NO: 2, or an amino acid sequence comprising a deletion, substitution or addition of one or several amino acids in said amino acid sequence and having a gonad-stimulating activity.

3: The invertebrate-derived gonadotropic hormone of claim 1, wherein said two peptides are obtained by purifying (a) a nerve organ removed from an invertebrate or (b) a certain organ reported to have gonad-stimulating activity, based on invertebrate gonad-stimulating activity as an indicator.

4: An isolated DNA coding any one of the following two peptides:
(a) A peptide having an amino acid sequence of SEQ ID NO: 1, or an amino acid sequence comprising a deletion, substitution or addition of one or several amino acids in said amino acid sequence and having a gonad-stimulating activity,
(b) A peptide having an amino acid sequence of SEQ ID NO: 2, or an amino acid sequence comprising a deletion, substitution or addition of one or several amino acids in said amino acid sequence and having a gonad-stimulating activity.

5: An isolated DNA, which comprises DNA coding the following two peptides and is more than 70% homologous to the nucleotide sequence of SEQ ID NO: 3:
(a) A peptide having an amino acid sequence of SEQ ID NO: 1, or an amino acid sequence comprising a deletion, substitution or addition of one or several amino acids in said amino acid sequence and having a gonad-stimulating activity,
(b) A peptide having an amino acid sequence of SEQ ID NO: 2, or an amino acid sequence comprising a deletion, substitution or addition of one or several amino acids in said amino acid sequence and having a gonad-stimulating activity.

6: A method for preparing peptides with gonad-stimulating activity comprising culturing and growing a host transformed by a vector containing the two DNA of claim 4.

7: The method of claim 6 further comprising mixing and oxidizing the peptides obtained from the host or a culture medium of the host.
